(19) United States
(12) Patent Application Publication
(10) Pub. No.: US 2022/0338721 A1
(43) Pub. Date: Oct. 27, 2022

(54) DEVICES, SYSTEMS, AND METHODS FOR FLUORESCENCE IMAGING

(71) Applicant: Canon U.S.A., Inc., Melville, NY (US)

(72) Inventors: Aaron David Selya, Boston, MA (US); Lampros Athanasiou, Medford, MA (US)

(21) Appl. No.: 17/727,573

(22) Filed: Apr. 22, 2022

Related U.S. Application Data
(60) Provisional application No. 63/180,324, filed on Apr. 27, 2021.

(51) Int. Cl. A61B 1/04; A61B 1/06; A61B 90/00; A61B 90/30

(52) U.S. Cl. CPC A61B 1/043; A61B 1/0655; A61B 90/361; A61B 90/30

(57) ABSTRACT

Devices, systems, and methods perform optical-scanning operations to acquire fluorescence data values corresponding to fluorescence data collected from inside a bodily lumen. A processor receives the fluorescence data; calculates a threshold background fluorescence value based on a central tendency of at least part of the fluorescence data values; discards fluorescence data values that are lower than the threshold background fluorescence value, thereby creating corrected fluorescence data values; and generates an image of the bodily lumen based on the corrected fluorescence data values.

### CROSS-REFERENCE TO RELATED APPLICATIONS

This application is related to, and claims priority from, co-pending U.S. Provisional Application Ser. No. 63/180,324, filed Apr. 27, 2021, the content of which is herein incorporated by reference in its entirety.

### BACKGROUND

### Field of Disclosure

This application generally concerns medical devices, systems, and methods that perform intravascular fluorescence imaging in an accurate and efficient manner.

### Background Information

Some medical imaging systems use fluorescence imaging, such as near-infrared fluorescence (“NIRF”) or near-infrared autofluorescence (“NIRAF”) to enable visualization of molecular processes (e.g., biological processes in an organism). For example, intravascular fluorescence is a newly proposed in-vivo imaging technique that uses a catheter-based imaging system to acquire fluorescence images of a patient's vasculature for the diagnosis of vascular diseases. Imaging catheters employed in intravascular fluorescence imaging use a rotating imaging core (e.g., an optical fiber) that delivers the excitation beam and collects the emitted light under automated control of specialized imaging and data-processing equipment.

In intravascular imaging, the emitted light collected by the catheter can be characterized by wavelength, amplitude, or lifetime of the fluorescence signal to obtain information on the emitting fluorophore and generate a functional characteristic of the arterial wall and plaques. Fluorescence detection data of molecular processes is obtained by integrating over a short period of time the emitted intensity, and/or by analyzing the wavelength, amplitude, lifetime, spectral shape or combinations thereof of the emitted fluorescence. Intravascular fluorescence imaging can be enhanced by using complementary imaging modalities such as intracoronary optical coherence tomography (OCT) or intravascular ultrasound (IVUS) imaging to provide functional information in a morphological context. OCT generates images with high spatial resolution 10-20 microns (μm) but with relatively low depth of penetration 1-2 millimeters (mm). In addition, due to the highly scattering nature of OCT, saline solution or contrast flushing agents are required during imaging acquisition. Nevertheless, when combined with NIRAF, the resulting multimodality (OCT-NIRAF) catheter provides complementary data on plaque structure and biological processes. See, for example, H. Wang, et al., “Ex vivo catheter-based imaging of coronary atherosclerosis using multimodality OCT and NIRAF excited at 633 nm,” Biomedical Optics Express 6(4), 1363-1375 (2015). The IVUS imaging modality is also widely used due to a better depth of penetration 5-8 mm, and no need for blood clearance (due low backscatter from blood) at 20 and 50 megahertz (MHz) radiation. But IVUS suffers of limited spatial resolution 100-250 μm, and requires relatively large probe sizes not adequate for use in most coronary arteries.

Therefore, an OCT-NIRAF catheter system continues to be developed due to its contrast-free autofluorescence method of acquiring intravascular images where the NIRAF signal can be correlated with vulnerable features like fibroatheroma, plaque rupture and in-stent restenosis. See, for example, Ughi G., et al., “Clinical characterization of coronary atherosclerosis with dual-modality OCT and near-infrared autofluorescence imaging”, JACC Cardiovasc Imaging. (2016) 9:1304-14.

Accurate visualization of molecular processes and morphological features is sometimes hindered by autofluorescence originated from unwanted sources. Autofluorescence from unwanted sources, which will be referred hereinafter as background fluorescence or background NIRAF, comes from a variety of sources, which can be classified into two main categories. The first is background fluorescence that is due to the instrument setup and imaging parameters, for example, autofluorescence from components of the imaging device (e.g., fluorescence from the optical fiber, lens, sheath, etc.), and ambient light (e.g., light from the operating room, display screens, etc.). The second source of background fluorescence is due to autofluorescence that does not originate from the desired targets.

More specifically, tissue components such as red blood cells (RBCs) and collagen are strongly fluorescent, which makes it difficult to discern between fluorescence from plaque and autofluorescence from blood, for example. When imaging media (e.g., staining solutions or flushing agents) are used, the fluorescence resulting from fluorophores not bound to specific target molecules also makes it difficult to distinguish between true fluorescence signals originated from plaque versus background fluorescence. In terms of instrument setup, fluorescence spectrometers detect intensity in counts-per-second (cps) but the measured intensity (in cps) can be different depending on the environment where the instrument is used or depending on the sensitivity of the instrument used. Also the intensity of detected fluorescence will depend on system parameters, such as excitation power, numerical aperture of the imaging system, camera noise, etc. The most common result of background fluorescence is an inconvenient random increment in signal levels that is hard to circumvent. Background fluorescence meddles with the detection of true fluorescence emitted by the fluorophore of interest, and complicates the detection of weak fluorescence signals.

Endogenous and exogenous fluoresce emission of biological tissue is related to several parameters of the excitation beam including wavelength, pulse duration, power density, etc. Until recently, endogenous fluorescence of tissue was neglected and often considered as a source of noise in medical imaging. However, new imaging modalities in medical devices have determined that endogenous fluorescence is a powerful technique for monitoring changes in biochemical structure, function, and composition of tissue without the potential perturbation or toxicity of external labels such as contrast agents or tagging solutions. However, endogenous fluorescence has low specificity and weak signals that are frequently signal-to-noise or background limited. Therefore, there are several known methods for removing background fluorescence, but new devices, systems, and methods for removal of background fluorescence continue to be developed.

A method of background fluorescence removal includes wavelength-based filtering. It involves the use of spectral filters (often placed in a filter wheel). The nature of the filters selected (cut-off wavelength region) is determined by the intended target fluorophore, and the overlap of its emission with the background fluorescence. It is most successfully applied when the spectral signatures of the targeted signal(s) as well as the undesired light are known. Since filters with limited blocking capability ultimately reduce image fidelity, wavelength-based filtering is often not effective. Another method of background fluorescence removal includes temporal filtering. This approach takes advantage of the short fluorescence lifetimes of organic fluorophores (e.g., in the order of nanoseconds (ns)), which is in contrast to much larger fluorescence lifetimes of inorganic materials (e.g., in the ordered of microseconds (μm) to milliseconds (ms)). This means that fluorescence from an inorganic material (e.g., contrast agent) can be observed “long” after the excitation and tissue autofluorescence has stopped. However, this requires a technically more advanced experimental setup and the use of contrast agents. See, e.g., Journal of Applied Physics, “The role of tissue fluorescence in in vivo optical bioimaging”, published 4 Nov. 2020, and U.S. patent Ser. No. 10/080,484 “Multispectral wide-field endoscopic imaging of fluorescence”, published 25 Sep. 2018.

Amore popular method of removing background fluorescence is based on numerical methods implemented by image processing. Digital processes for background fluorescence removal include digital filtering, fitting functions, entropy minimization, low rank method, morphological filtering, Gaussian blurring, rolling-ball algorithms, among others. See, for example, Yang, L. et al. “Fast Background Removal in 3D Fluorescence Microscopy Images Using One-Class Learning.” MICCAI (2015); Angelique Ale et al., “Background fluorescence subtraction technique for hybrid fluorescence molecular tomography/x-ray computed tomography imaging of a mouse model of early stage lung cancer”, Journal of Biomedical Optics 18(5), 056006 (May 2013); Profio, et al., “Digital background subtraction for fluorescence imaging”, Med Phys. 1986 September-October; 13(5):717-21; and Cao, A., et al., “A robust method for automated background subtraction of tissue fluorescence”, J. Raman Spectrosc., 38: 1199-1205, (2007).

Unfortunately, many of such numerical methods are directed to fluorescence microscopy, where fluorescent substances are examined by a microscope focusing light on a specimen repeatedly on a single plane at a time, and thus suffer from long computational time. Therefore, there is a need for devices, systems, and methods that perform intravascular fluorescence imaging with improved removal of background fluorescence. In particular, a new method for fluorescence suppression is highly desirable for intravascular multimodal imaging catheters where data collected by a large number helicoidally moving scans needs to be processed rapidly.

### SUMMARY

Some embodiments of a device for intravascular imaging comprise one or more computer-readable media storing instructions and one or more processors that are in communication with the one or more computer-readable media and that, when executing the instructions, cooperate with the one or more computer-readable media to cause the device to perform operations that comprise: obtaining detected fluorescence values of a bodily lumen; calculating a threshold background fluorescence value based on a central tendency of the detected fluorescence values; adjusting the detected fluorescence values based on the threshold background fluorescence value, thereby generating adjusted fluorescence values; and generating an image based on the adjusted fluorescence values.

According to one embodiment, a catheter-based imaging system for accurately quantifying fluorescence emission from a bodily lumen comprises: an imaging catheter that scans a bodily lumen with light of wavelength capable of stimulating emission of fluorescence light from within the bodily lumen; and a processor configured to: collect a plurality of fluorescence data values corresponding to the fluorescence light emitted from different regions within the bodily lumen; groups the plurality of fluorescence data values into one or more frames; calculates a threshold background fluorescence value based on a central tendency of one of the one or more frames; adjusts the fluorescence data values of the one or more frames based on the threshold background fluorescence value, thereby generating adjusted fluorescence data values; and generates an image based on the adjusted fluorescence data values.

According to one embodiment, a method for accurately quantifying fluorescence emitted from within a bodily lumen comprises: scanning a bodily lumen with an imaging catheter that transmits light of wavelength capable of stimulating emission of fluorescence light from within the bodily lumen; collecting a plurality of fluorescence data values corresponding to the fluorescence light emitted from different regions within the bodily lumen; grouping the plurality of fluorescence data values into one or more frames, calculating a threshold background fluorescence value based on a central tendency of one of the one or more frames; adjusting the fluorescence data values of the one or more frames based on the threshold background fluorescence value, thereby generating adjusted fluorescence data values; and generating an image based on the adjusted fluorescence data values.

Some embodiments of one or more computer-readable storage media store instructions that, when executed by one or more computing devices, cause the one or more computing devices to perform operations comprising: scanning a bodily lumen with an imaging catheter that transmits light of wavelength capable of stimulating emission of fluorescence light from within the bodily lumen; collecting a plurality of fluorescence data values corresponding to the fluorescence light emitted from different regions within the bodily lumen; grouping the plurality of fluorescence data values into one or more frames, calculating a threshold background fluorescence value based on a central tendency of one of the one or more frames; adjusting the fluorescence data values of the one or more frames based on the threshold background fluorescence value, thereby generating adjusted fluorescence data values; and generating an image based on the adjusted fluorescence data values.

These and other aspects and advantages of the present disclosure will be better appreciated by reference to the following drawings and detailed description.

### BRIEF DESCRIPTION OF THE DRAWINGS

FIG. 1 illustrates an example embodiment of a medical-imaging system that acquires intravascular images using a multimodality imaging catheter.

FIG. 2 illustrates an example embodiment of an operational flow for intravascular fluorescence imaging or OCT-NIRAF imaging executed by the medical-imaging system, according to the present disclosure.

FIG. 3 illustrates an example embodiment of an operational flow for calculating a background fluorescence value.

FIG. 4 illustrates an example embodiment of an operational flow for calculating a background fluorescence value.

FIG. 5 illustrates an example embodiment of an operational flow for calculating a background fluorescence value.

FIG. 6 illustrates a central tendency of detected fluorescence values on each frame from two optical-scanning procedures.

FIG. 7 illustrates an example embodiment of an operational flow for calculating a background fluorescence value.

FIG. 8 illustrates an example of sorted N detected fluorescence values from two optical-scanning procedures.

FIG. 9 illustrates an example embodiment of a medical-imaging system.

FIG. 10 illustrates an example of a Raw NIRAF data frame.

FIG. 11 illustrates an example of one or more image frames where background fluorescence removal was performed according to one or more algorithms based on a threshold background fluorescence value.

### DESCRIPTION

The following paragraphs describe certain explanatory embodiments. Other embodiments may include alternatives, equivalents, and modifications. Additionally, the explanatory embodiments include several novel features, and a particular feature may not be essential to some embodiments of the devices, systems, and methods that are described herein. Furthermore, some embodiments include features from two or more of the following explanatory embodiments. Embodiments described herein in context of the devices or systems are analogously valid for the respective methods, and vice versa. Furthermore, it will be understood that the embodiments described below may be combined, for example, a part of one embodiment may be combined with a part of another embodiment, or two or more embodiments may be combined.

Also, as used herein, the conjunction “or” generally refers to an inclusive “or,” although “or” may refer to an exclusive “or” if expressly indicated or if the context indicates that the “or” must be an exclusive “or.”

The terms “about” or “approximately” as used herein means, for example, within 10%, within 5%, or less. In some embodiments, the term “about” may mean within a measurement error. In this regard, where described or claimed, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range, if recited herein, is intended to be inclusive of end values and includes all sub-ranges subsumed therein, unless specifically stated otherwise. As used herein, the term “substantially” is meant to allow for deviations from the descriptor that do not negatively affect the intended purpose. For example, deviations that are from limitations in measurements, differences within manufacture tolerance, or variations of less than 5% can be considered within the scope of substantially the same. The specified descriptor can be an absolute value (e.g. substantially spherical, substantially perpendicular, substantially concentric, etc.) or a relative term (e.g. substantially similar, substantially the same, etc.).

Unless specifically stated otherwise, as apparent from the following disclosure, it is understood that, throughout the disclosure, discussions using terms such as “processing,” “computing,” “calculating,” “determining,” “displaying,” or the like, refer to the actions and processes of a computer system, or similar electronic computing device, or data processing device that manipulates and transforms data represented as physical (electronic) quantities within the computer system's registers and memories into other data similarly represented as physical quantities within the computer system memories or registers or other such information storage, transmission or display devices. Computer or electronic operations described in the specification or recited in the appended claims may generally be performed in any order, unless context dictates otherwise. Also, although various operational workflow diagrams are presented in a sequence(s), it should be understood that the various operations may be performed in other orders than those which are illustrated or claimed, or operations may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “in response to”, “related to,” “based on”, or other similar past-tense adjectives are generally not intended to exclude such variants, unless context dictates otherwise.

As it is known in the field of medical devices, the terms “proximal” and “distal” are used with reference to the manipulation of an end of an instrument extending from the user to a surgical or diagnostic site. In this regard, the term “proximal” refers to the portion (e.g., a handle) of the instrument closer to the user, and the term “distal” refers to the portion (tip) of the instrument further away from the user and closer to a surgical or diagnostic site. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

The term “autofluorescence” is used synonymously with NIRAF, and is meant to distinguish intrinsic fluorescence of fluorophores, cells or tissues from the fluorescence obtained by treating specimens with exogenous fluorescent markers that bind to such structures or act as contrast agents. As used herein, the term “background fluorescence” is used to mean fluorescence emanating from an unwanted source upon excitation with a light of wavelength capable of stimulating emission of fluorescence from within the bodily lumen. In one aspect, the wavelength of the excitation light source is selected to stimulate fluorescence emission from plaque, so that a method as described herein reduces autofluorescence of wavelengths in the range from the near infrared to the near ultraviolet.

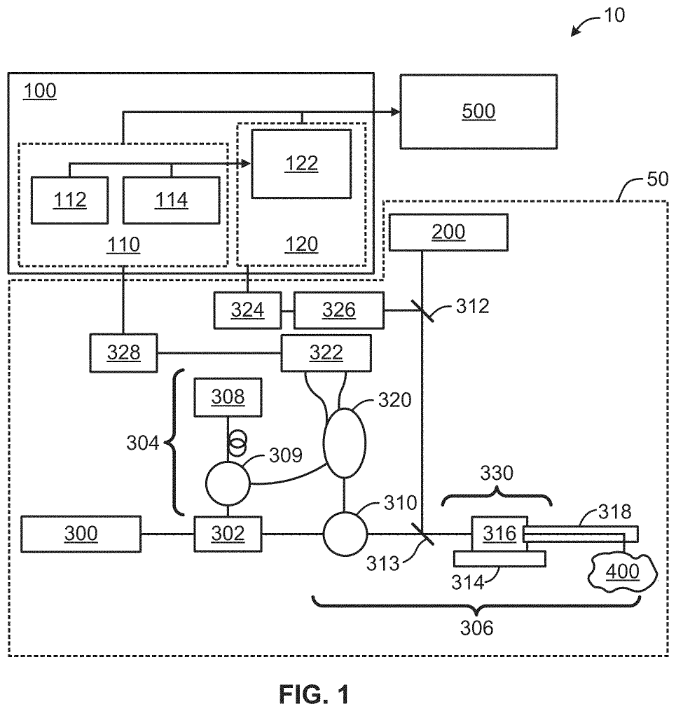

FIG. 1 illustrates in block diagram an example embodiment of a medical-imaging system. This embodiment of the medical-imaging system 10 can perform both optical-coherency tomography (OCT) imaging and fluorescence imaging (multimodality imaging). Other embodiments of the medical-imaging system 10 can perform only fluorescence imaging or only OCT imaging. Also, some embodiments of the medical-imaging system 10 may perform other modalities of imaging in addition to, or in alternative to, OCT imaging. The medical-imaging system 10 includes an imaging subsystem 50, which in turn may include at least one interferometer.

In the imaging subsystem 50, a sample arm 306 of the interferometer includes a patient interface unit (PIU) 330 and an optical-imaging device 318. In this embodiment, the optical-imaging device 318 includes a catheter that surrounds an imaging core. The imaging core includes optical components that can be rotated by a torque source arranged in the PIU. For example, some embodiments of the optical-imaging device 318 include a catheter with a double clad fiber (DCF) inside the catheter lumen and distal optics (e.g., a polished ball lens) at the tip of the fiber for side-view scanning. The distal optics of the optical-imaging device 318 may also include, a GRIN lens, a spacer (a portion of DCF), and a beam directing component (e.g., a mirror or prism or grating). The PIU 330 includes a fiber optic rotary junction (FORJ) 316 and a pullback unit 314. The rotary junction 316 may include a rotary motor or other torque source, and the pullback unit 314 may include a linear stage that can be powered by a linear piezoelectric transducer, for example. The rotary junction 316 rotates the imaging core inside the catheter to perform optical-scanning procedures inside a bodily lumen (e.g., vessel, bronchus, intestine, trachea, ear canal) or a bodily volume (e.g., stomach, nasal cavity). A specific example of optical-scanning procedure is an intravascular imaging procedure with is performed using a specialized catheter configured to be inserted into the vasculature of a patient. Here, the term “vasculature” refers to blood vessels, such as veins and arteries, in the body or in an organ or body part. Although portions of this disclosure may refer to veins and arteries, this disclosure is applicable to any type of blood vessel within the “vascular system.”

During an optical-scanning procedure, excitation light generated by an excitation-light source 200 is guided to the rotary junction 316, and then to the distal end of the optical-imaging device 318 to irradiate a sample 400 (e.g., an organ, tissue). In one embodiment, excitation light having a center wavelength of approximately 635 nm from a laser diode is coupled into a single mode fiber to deliver the excitation light to the PIU 330 via a non-illustrated cable bundle. The excitation light incident on the sample 400 causes the sample 400 to emit fluorescence light. The emitted fluorescent light has a wavelength longer than the wavelength of the excitation light. For example, if the excitation light has a first wavelength is in the range of 650 nm to 800 nm, the fluorescent light has a second wavelength in a range of 700 nm to 850 nm.

In some embodiments, the fluorescence light generated by the sample 400 includes autofluorescence light, which is the endogenous fluorescence light that is generated without application of a dye or an agent. And, in some embodiments, the fluorescence light generated by the sample 400 may include fluorescence light generated by an exogenous fluorescent dye or agent added (e.g., intravenously) to the sample 400. The fluorescence light emitted from the sample 400 is collected by the cladding of the double clad fiber of the optical-imaging device 318 and provided to a fluorescence detector 326. In one embodiment, the fluorescence detector 326 is a photomultiplier tube (PMT) optimized to have better signal-to-noise ratio (SNR) than other detectors (e.g., CCD camera). For example, the PMT gain is calibrated to have consistent detection sensitivity. The fluorescence detector 326 generates fluorescence-detection data that include detected values of the fluorescence light (detected fluorescence values). The detected fluorescence values may indicate the intensities of the detected fluorescence light. The fluorescence detector 326 digitizes the detected fluorescence values, and provides the fluorescence-detection data, which include the detected fluorescence values, to an image-processing device 100 via a first data-acquisition device 324.

Additionally, an OCT light source 300 generates OCT light (e.g., light with a wavelength of around 1.3 μm), which is delivered to a splitter 302. The splitter 302 splits the OCT light into a reference arm 304 and the sample arm 306, e.g., in a 50/50 ratio. A reference beam from the OCT light traveling along the reference arm 304 is reflected from a reference mirror 308, while a sample beam from the OCT light traveling along the sample arm 306 (through the core of the double clad fiber of the optical-imaging device 318) is incident on the sample 400, and is reflected and/or scattered by the sample 400. Some of the reflected or scattered OCT light travels through the double clad fiber of optical-imaging device 318, the rotary junction 316, and a circulator 310, and is delivered to a coupler 320.

In the coupler 320, the OCT light reflected from the reference mirror 308 and the OCT light reflected or scattered from the sample 400 are coupled to generate interference patterns. Interference patterns are generated only when the optical path length of the reference arm is equal to the optical path length of the sample arm to within a coherence length of the light source 300. The interference patterns are detected by an OCT detector 322, such as a photodiode or a multi-array camera. The interference patterns are delivered to the image-processing device 100 through a second data-acquisition device 328. In some embodiments, the OCT interference patterns and the fluorescence-detection data are delivered to the image-processing device 100 concurrently or simultaneously. In this manner, the image processing device 100 can use the OCT interference patterns and the fluorescence-detection data to generate co-registered fluorescence and OCT images of the sample 400.

In this embodiment, the image-processing device 100 includes an OCT unit 110 for collecting and processing OCT data, and includes a fluorescence unit 120 for collecting and processing fluorescence-detection data. The OCT unit 110 includes a unit 112 for optical-attenuation-property calculation and a unit 114 for calculating distance between the sample 400 and the exit end of the optical-imaging device 318 from which the OCT light is incident on the sample 400. The optical-attenuation property and the distance calculated by the OCT unit 110 are output to the fluorescence unit 120. Based on the calculated optical-attenuation property and the calculated distance, the fluorescence unit 120 may calibrate the detected fluorescence values. Distance calibration based on detected fluorescence values has been described by the applicant of the present application in previous disclosures including, for example, U.S. Pat. Nos. 10,952,616 and 11,147,453, and pre-grant patent application publication US 20210121132, the disclosures of which are herein incorporated by reference.

In some embodiments, information that correlates with the fluorescence-collection efficiency, other than an optical-attenuation property of the sample 400, is used instead of the distance between the optical-imaging device 318 and the sample 400. The information may correlate the acquired data with the distance between the optical-imaging device 318 and the sample 400. For example, the instrument's limit of detection (LOD) for detecting intensity of the light reflected from the sample 400 may be used to calibrate the detected fluorescence values. The LOD can be defined by the lowest concentration of fluorescence that can be distinguished by the detector 326.

During an optical-scanning procedure, the position of an optical waveguide inside the optical-imaging device 318 can be adjusted or controlled by a pullback unit 314 to scan the sample 400 at least one full revolution (i.e., 360 degrees), while simultaneously moving linearly backwards in a direction from the distal to the proximal end. In some embodiments, the rotary junction 316 is located on the pullback unit 314. The medical-imaging system 10 further includes a first dichroic filter 312 and a second dichroic filter 313 for separating and directing the OCT light, the excitation light, and the fluorescence light to the desired locations or devices. The rotary junction 316 can rotate the imaging core inside the optical-imaging device 318 to obtain cross-sectional images of the bodily lumen or bodily volume. The imaging core can be simultaneously moved longitudinally during the rotation so that optical-scanning data (e.g., OCT data, and/or fluorescence-detection data) are obtained in a helical scanning pattern. For example, the rotation and translation movements can helically scan the inside a bodily lumen by light emitted and collected by imaging core. Based on the light collected by the imaging core, the fluorescence detector 326 and the OCT detector 322 provide data to the processor 100, which produces a series of adjacent helical A-scans of the bodily lumen, which can then be used to create a two-dimensional (2D) tomogram called a B-scan. Also for example, moving the imaging core longitudinally within the bodily lumen allows the collection of a series of B-scans, which can be combined to form a three-dimensional (3D) image of the bodily lumen.

Based on the scan data (e.g., OCT data, fluorescence-detection data) that are obtained during an optical-scanning procedure, the image-processing device 100 generates a fluorescence image, an OCT image, or a multi-modal image, such as an OCT-fluorescence image (e.g., a co-registered OCT-fluorescence image), and the image-processing device 100 provides the image to a display device 500, which displays the image. The image-processing device 100 can control the display device 500 to display the resulting image or images in various formats. Examples of image display are described in previous disclosures of the applicant, for example, in U.S. patent Ser. No. 11/145,054 or pre-grant patent application publication US 20190339850, which are hereby incorporated by reference.

Here, it must be kept in mind that a rotating imaging core scans the physical space of a bodily lumen (e.g., airways and blood vessel lumens) in polar coordinates (e.g., radius and angle). Therefore, the B-scan images of a bodily lumen are generally displayed in polar coordinates (i.e., as radar-like images), where the radius represents the depth of imaging (distance from the catheter axis to the lumen wall) and the circumference represents approximately one full revolution (a 360-degree scan) of the imaging core.

However, the acquired B-scan image must be digitally represented (displayed) in arrays of pixels which are inherently rectangular. Therefore, polar images are converted from their rectangular representation before displaying to the viewer on a display device. Since quantitative values (e.g., lumen diameters, lumen areas, circumferences, etc.) are to be measured on the polar image, the transformation from rectangular-to-polar or vice versa must preserve relative distances between pixels in all dimensions (radial and angular). Generally, the depth of imaging in a B-scan (y axis in rectangular coordinates) maps directly to the radius of the image, and the circumference in the B-scan (x axis in rectangular coordinates) maps to the polar angle (some increment of 2π radians or 360 degrees).

For example, when a normalized rectangular image of a lumen in x, y Cartesian coordinates is converted to a normalized polar image in r, θ polar coordinates, y=0 (e.g. the bottom row of the rectangular image) maps to radius r=0 (the center of the polar image), and y=1 (the top row of the rectangular image) maps to radius r=1 (the outer diameter of the polar image). Likewise, x=0 (the left column in the rectangular image) maps to angle 0=0 degrees and x=1 maps to approximately 359 degrees of the polar image. Here, the maximum value of x (i.e., x=1) may not map to an exact complete revolution because the imaging core is rotated and translated.

For accurate quantitative dimensional measurement in polar images, pixels mapping to radius r=0 should represent the actual physical space at the center of the axis of rotation of the imaging core, otherwise the polar image will be expanded or contracted in the radial direction. However, when the catheter is scanning a bodily lumen of a tortuous path (e.g., when the catheter is bent and the imaging core does not remain in the center of the catheter lumen), the pixels at y=0 do not necessarily correspond to r=0, and must be shifted in the y-dimension until such correspondence is satisfied before mapping to a polar representation. This is particular true when the optical path length of the sample and reference arms of the interferometer are not equal. For example, when the catheter is pushed or pulled longitudinally during scanning a bodily lumen, the optical fiber in the imaging core can be compressed or stretched and thus an optical path length displacement occurs. A method to address optical path length displacement is known as z-offset calibration, and generally comprises automatically detecting the displacement effect by searching for image features that are stationary (not changing), and calibrating successive scan data to such image features so that polar representations of a bodily lumen can be used for accurate dimensional measurements (e.g., accurate lumen diameters, lumen areas, circumferences, etc.).

In the resulting multimodality image, the spaces with NIRAF signal should have strong correspondence with spaces where OCT data shows the existence of plaque. This strong OCT-NIRAF data correspondence can assist doctors in determining areas of plaque buildup in vulnerable patients. However, when background fluorescence is not removed or suppressed, the characterization of the fluorescent tissue becomes inaccurate which can lead to false positive or false negative determinations. Therefore, the inventors herein propose a novel technique whereby background fluorescence is suppressed, removed, or at least minimized in raw data acquired directly from the detector.

Factors that influence the detected fluorescence values (which indicate the perceived intensity of the fluorescence light) include the device that is used to detect the fluorescence light and the properties of the environment in which the fluorescence light is detected. To accurately determine the detected fluorescence values of a sample, the image-processing device 100 calculates the background fluorescence values, and subtracts the background fluorescence from the detected fluorescence values. Background fluorescence can be a portion (e.g., a percentage) of the perceived autofluorescence intensity, or a part of the shape of the autofluorescence spectrum which can be quantified according to known normalization techniques.

Conventionally, as discussed above, fluorescence-background-value calculation for an optical-imaging device can be performed by placing a fluorescence-light-proof sheath around the optical-imaging device prior to using the device in a subject. A reading is taken in this non-fluorescence environment, and the background fluorescence value is calculated based on the reading. Once the detected fluorescence values are collected in vivo, the background fluorescence value is subtracted from the detected fluorescence values to reveal the fluorescence values independent of the background. However, this requires a user to perform work (e.g., place the fluorescence-light-proof sheath around the optical-imaging device) which is unrelated to the imaging procedure and to properly use additional equipment. Furthermore, the calculation of the background value is done using data collected outside of the environment in which the instrument is going to be used, which may increase the risk that the background-value calculation is inaccurate.

Thus, the medical-imaging system 10 according to the present disclosure includes several improvements, such as the following: The medical-imaging system 10 can calculate the background fluorescence value during the course of normal imaging in a manner that can be invisible to the user (does not require any additional work from the user and the user may even be unaware of the calculation). The medical-imaging system 10 can calculate the background fluorescence value from within the environment where the optical scanning is being performed. And the improved calculation of the background fluorescence value can allow the medical-imaging system 10 to generate images that more accurately depict the details and dimensions of the sample, which can be used to improve the diagnosis and treatment of a patient.

In the MMOCT application a typical pullback operation can result in about 200,000 individual NIRAF sample values. Within this large number of samples, there is a significant number of NIRAF values that represent the underlying background fluorescence in an image. Since it is unpractical to examine all individual sample values to remove the background fluorescence, the inventors herein have developed several algorithms that can calculate the background fluorescence using only part of the collected data. At least one of these algorithms is specific to our MMOCT application's implementation of data collection, but all algorithms provide a novel technique that can be applied to other imaging catheters. In our MMOCT application, NIRAF values are collected at a rate of 1 value per A-line and grouped into frames consisting of 500 A-lines. A single frame represents the data collected from one full rotation of our imaging catheter used in the MMOCT application. To determine the background within the environment where the data is collected, an average NIRAF value is calculated for each frame. This is done by recording the sum of all the NIRAF values and diving the sum by the number of NIRAF values in a frame (500). Then all of the frames within the procedure are compared to each other, and the frame with the lowest value among all frames is designated as a background threshold. The use of an average across a 500 A-line sample frame as the value limits the possibility for A-lines with artificially high or artificially low values to impact the final calculation, and completely removes regions with higher values than the background from consideration in the calculation of the background threshold.

In the case that MMOCT data collection is performed in a different manner, alternative approaches can be used for background fluorescence removal. In one embodiment, the system can take the average of fluorescence values collected in the first frame (500 samples). Despite the small sample size this technique works because during normal use of the MMOCT application the user will typically begin data collection in an area that has low NIRAF (e.g. distal to stenosis). This avoids the need of calculating background in a different environment than the operating environment. Notably, this technique relies on the user to provide an initial starting point which is representative of the background environment. So while this can be used provide enough data to get a good approximation of the background value a larger sample size is preferred for accuracy. Therefore, to obtain a more accurate result, the inventors propose calculating a background fluorescence threshold using a portion of the lowest values from across all samples of a full pullback procedure. For example, as mentioned above, a typical pullback operation will result in bout 200,000 individual NIRAF sample values. To minimize the calculation time, the present disclosure uses about 1% (2000) lowest values of the total values collected in a typical MMOCT operation. Using the average of NIRAF values of the lowest 2000 samples in the operation gets same results as using the lowest average NIRAF value across the frames of the entire operation, but significantly reduces the processing time.

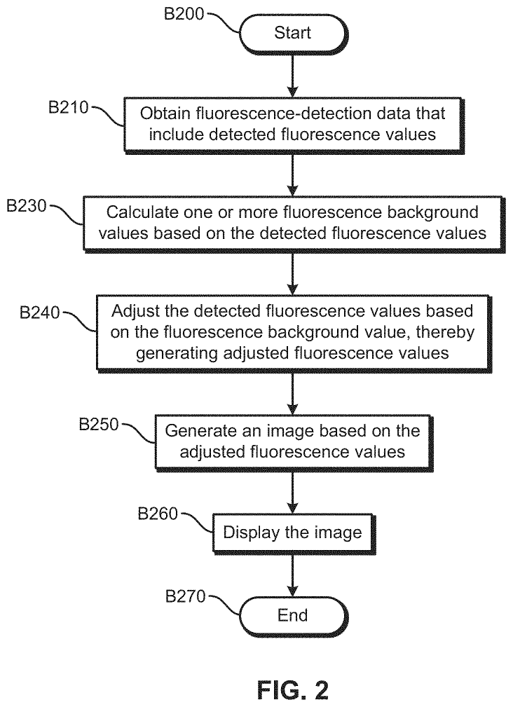

FIG. 2 illustrates an example embodiment of an operational workflow for intravascular fluorescence imaging and background removal. Although this operational flow and the other operational flows that are described herein are each presented in a certain order, some embodiments of these operational flows may perform at least some of the operations in different orders than the presented orders. Examples of different orders include concurrent, parallel, overlapping, reordered, simultaneous, incremental, and interleaved orders. Thus, other embodiments of the operational flows that are described herein may omit blocks, add blocks, change the order of the blocks, combine blocks, or divide blocks into more blocks. Similarly, although the workflow illustrates fluorescence imaging, it will be appreciated that the operational workflow of FIG. 2 may include simultaneous intravascular OCT and intravascular fluorescence imaging.

In FIG. 2, the workflow starts in block B200. For example, at block B200, the medical-imaging system 10 is set in an active mode (turned ON), and the respective subsystems are calibrated for use. Then the flow moves to block B210, where the medical-imaging system obtains fluorescence-detection data that include detected fluorescence values. For example, the imaging subsystem 50 in FIG. 1 may illuminate a sample 500 with excitation light and generate detected fluorescence values based on detected fluorescence light. The fluorescence-detection data, which include the detected fluorescence values, are obtained by the image-processing device 100.

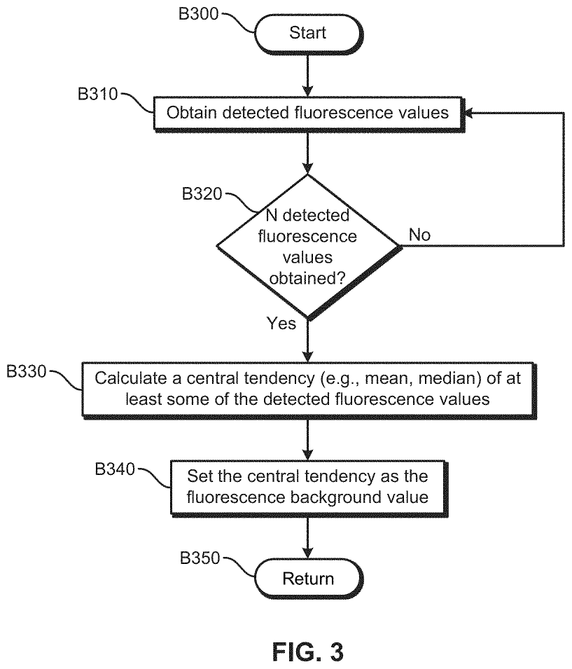

Next, in block B230, the image-processing device 100 calculates one or more background fluorescence values based on the detected fluorescence values (e.g., as described in FIGS. 3-5 and 7). Then, in block B240, the image-processing device 100 adjusts the detected fluorescence values based on one of the one or more background fluorescence values, thereby generating adjusted fluorescence values. For example, in some embodiments, the image-processing device subtracts a background fluorescence value from each of the detected fluorescence values. In other embodiments, the image processing device removes (discards) detected fluorescence values that are equal to or lower than a threshold background fluorescence value.

The flow then moves to block B250, where the image-processing device 100 generates an image based on the adjusted fluorescence values. At block B250, the image-processing device 100 may generate an image of the sample showing the detected fluorescence values. In block 260, the image-processing device provides the image to a display device, which displays the image. Finally, the flow ends in block B270.

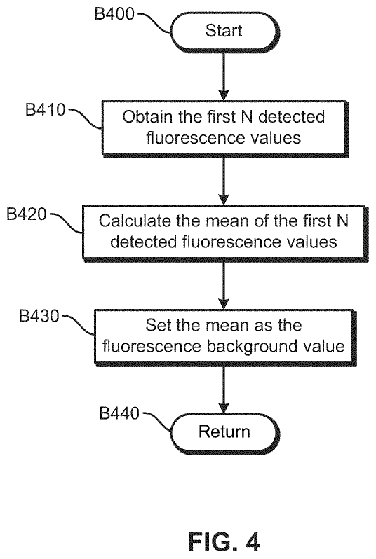

FIG. 3 illustrates an example embodiment of an operational flow for calculating a threshold background fluorescence value. Furthermore, although this operational flow and the operational flows in FIGS. 4, 5, and 7 are performed by an image-processing device, some embodiments of these operational flows can be performed by two or more image-processing devices or by one or more of other specially-configured (e.g., distributed) computing devices.

In FIG. 3, the flow begins in block B300 and moves to block B310, where the image-processing device 100 obtains detected fluorescence values, for example, from the imaging subsystem 50, or from a local or remote storage. In some embodiments, the image-processing device obtains the detected fluorescence values from an imaging subsystem while a catheter device of the imaging subsystem 50 is detecting fluorescence light from inside a bodily lumen or bodily volume, and the catheter device may be moving while detecting the fluorescence light. In other embodiments, the detected fluorescence values can be pre-stored in a local or remote storage. After the image-processing device 100 obtains detected fluorescence values, the flow moves to block B320, where the image-processing device determines whether N detected fluorescence values have been obtained, where N is a positive integer greater than 2. For example, in some embodiments, N is 500, 1,000, or 2,000. Also, in some embodiments, N is equal to a certain percentage of the total number of detected fluorescence values that are obtained in a typical optical-scanning procedure. And, in some embodiments, N is equal to the number of detected fluorescence values in one frame. If the image-processing device determines that N detected fluorescence values have not been obtained (B320=No), then the flow returns to block B310. If the image-processing device determines that N detected fluorescence values have been obtained (B320=Yes), then the flow proceeds to block B330. In a MMOCT application, fluorescence values may be collected at a rate of 1 value per A-line and grouped into B-scan frames consisting of N A-lines per frame. A single B-scan frame represents the data collected from one full rotation of the imaging core arranged inside the catheter.

In block B330, the image-processing device calculates a statistical central tendency (e.g., a mean, a median, a mode) of at least some of the detected fluorescence values. As used herein, the mean refers to the arithmetic mean found by adding the values (e.g., intensity) of N detected fluorescence measurements, and dividing the sum by the N number of measurements; this is often known colloquially as an average. The median is the middle value in a list ordered values from smallest to largest. The mode is the most frequently occurring value on a list of values. As noted above, N can be equal to a certain percentage of the total number of detected fluorescence values that are obtained in a typical optical-scanning procedure (e.g., a pullback procedure). And, in some embodiments, N is equal to the number of A-lines detected fluorescence values in one B-scan frame.

For example, in one embodiment, the image-processing device may calculate a mean of all of the detected fluorescence values, or the image-processing device may select a subset of the detected fluorescence values and calculate a median of the detected fluorescence values in the subset. In one example, to determine the background fluorescence within the environment (e.g., inside the bodily lumen or bodily volume), the central tendency (an average) fluorescence value is calculated for each frame. This is done by recording the sum of all of the detected fluorescence values, and diving the sum by the number N of fluorescence values in a frame. Then the frames within the procedure are iteratively compared to each other, and the frame with the lowest fluorescence value among all frames is designated as a threshold value for background removal. The use of an average as the central tendency in a sample frame limits the possibility that A-lines with artificially high or low fluorescence values may impact the final calculation, and completely removes regions with higher fluorescence values than the background from consideration in the calculation of the central tendency.

Next, in block B340, the image-processing device sets the central tendency as the threshold background fluorescence value. Finally, the flow ends in block B350. More specifically, the threshold background fluorescence value obtained at block B340 is returned to the flow of FIG. 2, and used at block B240 of FIG. 2.

Furthermore, in at least some embodiments, the image-processing device continues to obtain detected fluorescence values from an imaging subsystem while performing blocks B330-B340, and, in some embodiments, even after block B340 is completed.

FIG. 4 illustrates another example embodiment of an operational workflow for calculating a threshold background fluorescence value. This embodiment takes advantage of the tendency that users have to begin data collection using a catheter device in a region of a bodily lumen or bodily volume that emits little fluorescence light (e.g., an area that has low NIRAF). The flow begins in block B400 and moves to block B410, where an image-processing device obtains the first N detected fluorescence values (where N is a positive integer greater than 2). For example, in some embodiments, N is the first 200, 500, or 900 A-lines of a first B-scan frame. In some embodiments, N is equal to the number of detected fluorescence values (A-lines) in the first full frame, or N is equal to a multiple of the number of detected fluorescence values in a frame. Also, in some embodiments, N is equal to a certain percentage of the total number of detected fluorescence values that are obtained in a typical optical-scanning procedure. Furthermore, in some embodiments, the image-processing device obtains the N detected fluorescence values from an imaging subsystem while a catheter device of the imaging subsystem is detecting fluorescence light from inside a bodily lumen or bodily volume, and the catheter device may be moving while detecting the fluorescence light. At block B410, acquiring the first N detected fluorescence values in a region of low NIRAF allows the impact of fluorescence in a region of the artery without any plaque, representing a background fluorescence, to be taken into account in the process of background removal.

Next, in block B420, the image-processing device calculates the mean (or other central tendency) of the first N detected fluorescence values. The flow then moves to block B430, where the image-processing device sets the mean (or other central tendency) as the background fluorescence value. Finally, the flow ends in block B440. More specifically, the threshold background fluorescence value obtained at block B430 is stored for use at block B240 of FIG. 2. Furthermore, in some embodiments, the image-processing device continues to obtain detected fluorescence values from an imaging subsystem while performing blocks B420-B430, and, in some embodiments, even after block B430 is finished.

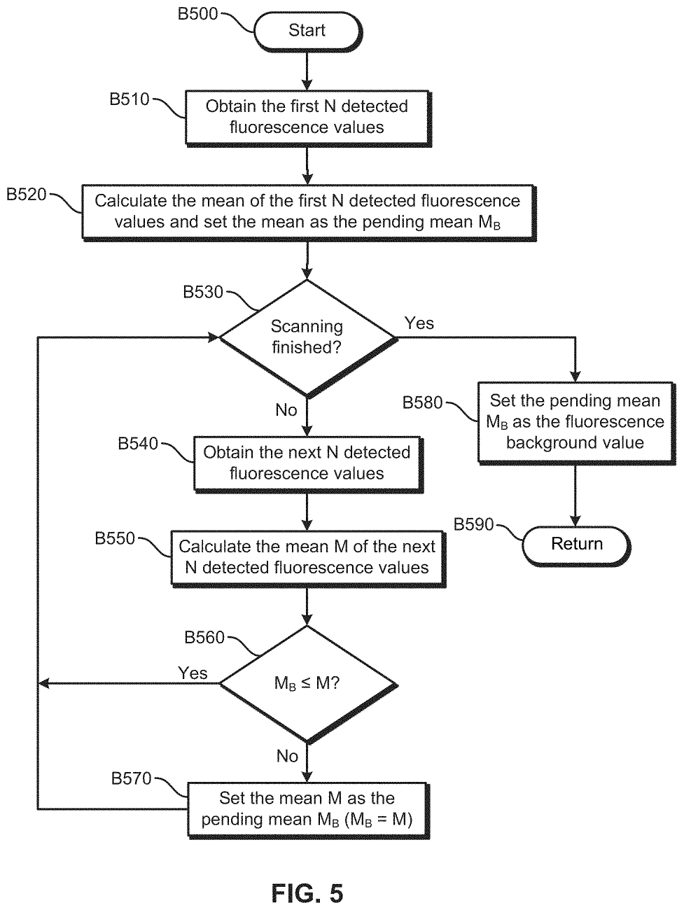

FIG. 5 illustrates another example embodiment of an operational flow for calculating a threshold background fluorescence value. The flow starts in block B500 and moves to block B510, where an image-processing device obtains the first N detected fluorescence values. In some embodiments, the image-processing device obtains the first N detected fluorescence values from an imaging subsystem while the imaging subsystem is performing an optical-scanning procedure, for example an optical-scanning procedure in which a catheter device of the imaging subsystem is detecting fluorescence light from inside a bodily lumen or bodily volume, and the catheter device may be moving while detecting the fluorescence light.

The flow then proceeds to block B520, where the image-processing device calculates the mean (or other central tendency) of the first N detected fluorescence values and sets the mean as a temporary or pending mean M_{B}.

The flow then proceeds to block B530, where the image-processing device determines if the optical-scanning procedure is finished (e.g., if an imaging subsystem continues to generate detected fluorescence values). If the image-processing device determines that the optical-scanning procedure is not finished (B530=No), then the flow moves to block B540. If the image-processing device determines that the optical-scanning procedure is finished (B530=Yes), then the flow proceeds to block B580.

In block B540, the image-processing device obtains the next N detected fluorescence values. The image-processing device may obtain the next N detected fluorescence values from the imaging subsystem while the imaging subsystem continues to perform the optical-scanning procedure.

Next, in block B550, the image-processing device calculates the mean M (or another central tendency) of the next N detected fluorescence values. The flow then proceeds to block B560, where the image-processing device determines whether the pending mean M_{B} is less than (or equal to) the mean M. If the image-processing device determines that the pending mean M_{B} is less than the mean M (B560=Yes), then the flow returns to block B530. If the image-processing device determines that the pending mean M_{B} is not less than (or equal to) the mean M (B560=No), then the flow moves to block B570. In block B570, the image-processing device changes the pending mean M_{B} by setting the mean M as the pending mean M_{B} (M_{B}=M). The flow then returns to block B530.

If, from block B530, the flow moves to block B580, then, in block B580, the image-processing device sets the pending mean M_{B} as the background fluorescence value. Finally, the flow ends in block B590. More specifically, at block B590, the pending mean M_{B} set as the background fluorescence value is used by the image-processing device at block B240 to adjust the detected fluorescence values. In this manner, the mean M of the next N detected fluorescence values is used as the background fluorescence value (background fluorescence threshold) to adjust the detected fluorescence values at block B240.

For example, in some embodiments of the medical-imaging system 10 in FIG. 1, detected fluorescence values are collected at a rate of one value per A-line and grouped into B-scan frames consisting of 500 A-lines. A single B-scan frame represents the data collected from one full rotation of the imaging core in the optical-imaging device 318. To determine the background fluorescence value within the imaging environment, the average detected fluorescence value is calculated for each frame. Thus, N is set to the number of A-lines in a frame, which is 500 in this example. Also, the respective central tendency of the detected fluorescence values of the frames are calculated (B520 and B550). The central tendencies are compared to each other, and the lower of the two is designated as the background fluorescence value (B560, B570, and B580). The same concept applies to any number of frames acquired in a pullback operation. The use of a lowest central tendency (e.g., the lowest mean) across multiple frames of a procedure limits the effect that A-lines with artificially high or artificially low detected fluorescence values can have on the final fluorescence-background-value calculation. This removes regions with higher values than the background from consideration in the calculation of the background fluorescence threshold.

In one example, we have used N=500 as that is the number of A-lines per OCT frame acquired by our MMOCT system. However, the algorithm would work as implemented even if there is only 1 A-line per frame. As long as we had at least 2 frames with the lower value frame serving as the background fluorescence value used as a threshold. This is true when the algorithm uses an average as the central tendency. In other words, in some embodiments N can be defined as “an integer greater than 2” where the “central tendency” can be either the average, median, or mode. In other embodiments, N can be defined as an integer equal to 2 where the central tendency is an average. In further embodiments, N is an integer in a range between 2 and the maximum of number of A-lines of one frame; or N is an integer in a range between 2 and the maximum number of frames in the entire pullback.

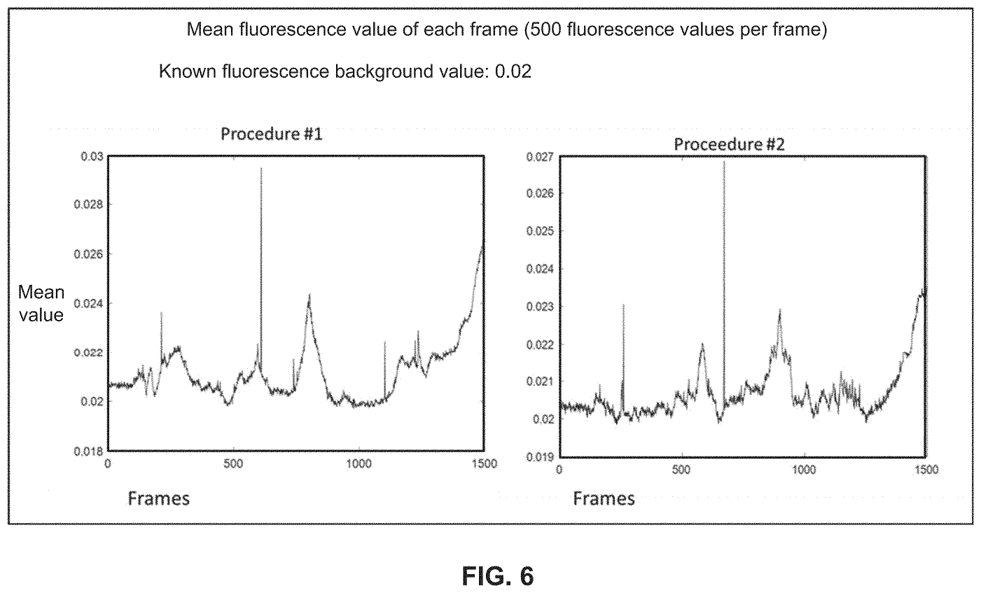

FIG. 6 illustrates an example of the mean of detected fluorescence values from two optical-scanning procedures. Here, the background fluorescence value in the two procedures was known or presumed to be approximately 0.02. The mean detected fluorescence values were calculated using the detected fluorescence values of each frame, and each frame included 500 respective detected fluorescence values. As shown by FIG. 6, if the lowest mean detected fluorescence value of procedure #1 is set as the background fluorescence value, then the background fluorescence value would be slightly above 0.02, which is the known background fluorescence value. On the other hand, if the lowest mean detected fluorescence value of procedure #2 is set as the background fluorescence value, then the background fluorescence value would be slightly below 0.02, which is the known background fluorescence value. Evidently, using the known or presumed background value could underestimate or overestimate the level (intensity) of the background fluorescence value. The various algorithms disclosed herein minimize the probability of background miscalculation.

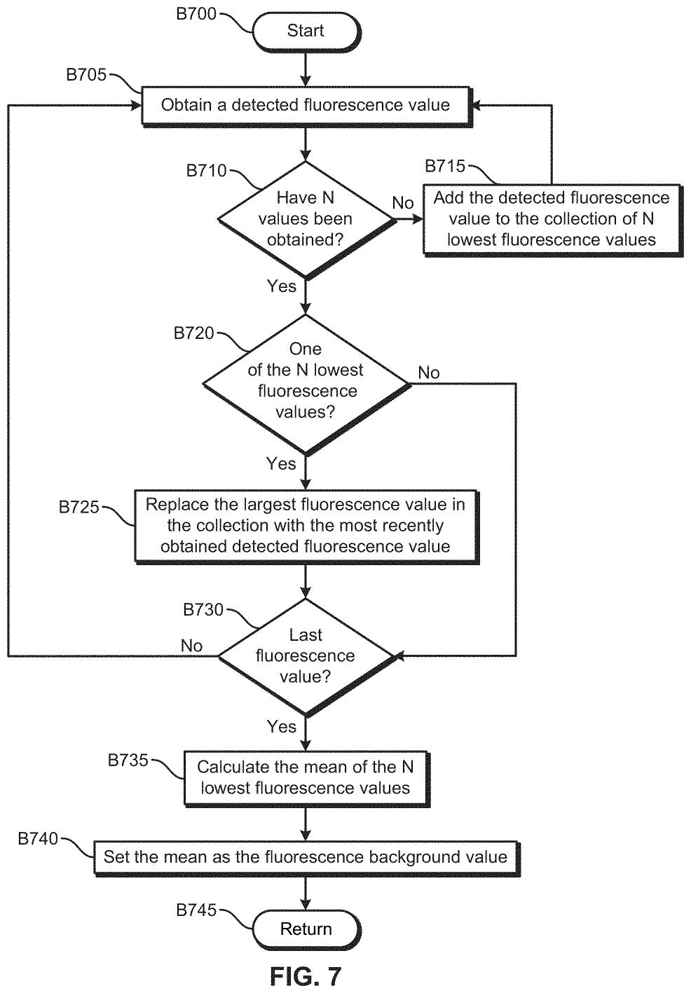

FIG. 7 illustrates another example embodiment of an operational workflow for calculating an adjusting threshold background fluorescence value. The workflow begins in block B700, where an image-processing device may perform initialization operations, for example clearing a collection of N lowest fluorescence values and resetting a counter of obtained detected fluorescence values.

Then the flow moves to block B705, where the image-processing device obtains a detected fluorescence value. In some embodiments, the image-processing device obtains the detected fluorescence values from an imaging subsystem while the imaging subsystem is performing an optical-scanning procedure, for example an optical-scanning procedure in which a catheter device of the imaging subsystem is detecting fluorescence light from inside a bodily lumen or bodily volume, and the catheter device may be moving while detecting the fluorescence light.

Next, in block B710, the image-processing device determines whether it has obtained N detected fluorescence values (where N is a positive integer). For example, in some embodiments, N is 1,000, 2,000, or 3,000. And, in some embodiments, N is equal to the number of detected fluorescence values in a frame, or a fraction of the number of detected fluorescence values in multiple frames, or a multiple of the number of detected fluorescence values in a frame. Also, in some embodiments, N is equal to a certain percentage of the total number of detected fluorescence values that are obtained in a typical optical-scanning procedure (e.g., a full pullback). For example, in some embodiments, 200,000 detected fluorescence values are obtained in a typical optical-scanning procedure, and N is equal to 1% of the total number of detected fluorescence values that are obtained in a typical optical-scanning procedure. Thus, in such embodiments, N is 2,000. Here, the 2,000 detected fluorescence values can correspond to the lowest of the 200,000 detected fluorescence values (e.g., 1 value out of each 100 detected fluorescence values) obtained in a typical optical-scanning procedure.

At block B710, if the image-processing device determines that it has not obtained N detected fluorescence values (B710=No), then the flow moves to block B715, where the image-processing device adds the detected fluorescence value to a collection (memory) of N lowest detection values. Following block B715, the flow returns to block B705 to again obtain a detected fluorescence value. If the image-processing device determines that it has obtained N detected fluorescence values (B710=Yes), then the flow moves to block B720.

In block B720, the image-processing device determines whether the most-recently-obtained detected fluorescence value is one of the N lowest detected fluorescence values. For example, the image-processing device may determine whether the most-recently-obtained detected fluorescence value is lower than the highest detected fluorescence value in the collection of N lowest fluorescence values. In some embodiments in which the collection of N lowest fluorescence values is a list that is sorted in order of lowest to highest, the image-processing device compares the most-recently-obtained detected fluorescence value to the detected fluorescence value at the end of the list. If the image-processing device determines that the most-recently-obtained detected fluorescence value is not one of the N lowest detected fluorescence values (B720=No), then the flow proceeds to block B730. If the image-processing device determines that the most-recently-obtained detected fluorescence value is one of the N lowest detected fluorescence values (B720=Yes), then the flow moves to block B725.

In block B725, the image-processing device replaces the largest detected fluorescence value in the collection of N lowest detected fluorescence values with the most-recently-obtained detected fluorescence value. For example, in some embodiments in which the collection of N lowest fluorescence values is a list that is sorted in order of lowest to highest, the image-processing device replaces the last detected fluorescence value in the list (the highest detected fluorescence value) with the most-recently-obtained detected fluorescence value, and then the image-processing device re-sorts the list. The flow then advances to block B730.

In block B730, the image-processing device determines whether the last detected fluorescence value has been obtained (e.g., whether an optical-scanning procedure has been completed, or whether a stop instruction has been received). If the image-processing device determines that the last detected fluorescence value has not been obtained (B730=No), then the flow returns to block B705 to again obtain a detected fluorescence value. If the image-processing device determines that the last detected fluorescence value has been obtained (B730=Yes), then the flow moves to block B735.

In block B735, the image-processing device calculates the mean (or other central tendency) of the N lowest detected fluorescence values. Then, in block B740, the image-processing device sets the mean (or other central tendency) as the background fluorescence value. Finally, the flow ends in block B745. Here too, the background fluorescence value obtained at block B740 is provided to block B240 of FIG. 2 to adjust the detected fluorescence values. As it can be appreciated from FIG. 7, after B730=YES, only the N lowest detected fluorescence values are used to set the background fluorescence value. In the case of obtaining additional detected fluorescence values while simultaneously calculating the fluorescence background value based on the previously detected fluorescence values, the additional detected fluorescence values are not used to calculate the fluorescence background value if the additional detected (most recently obtained) fluorescence values are higher than the previously obtained values.

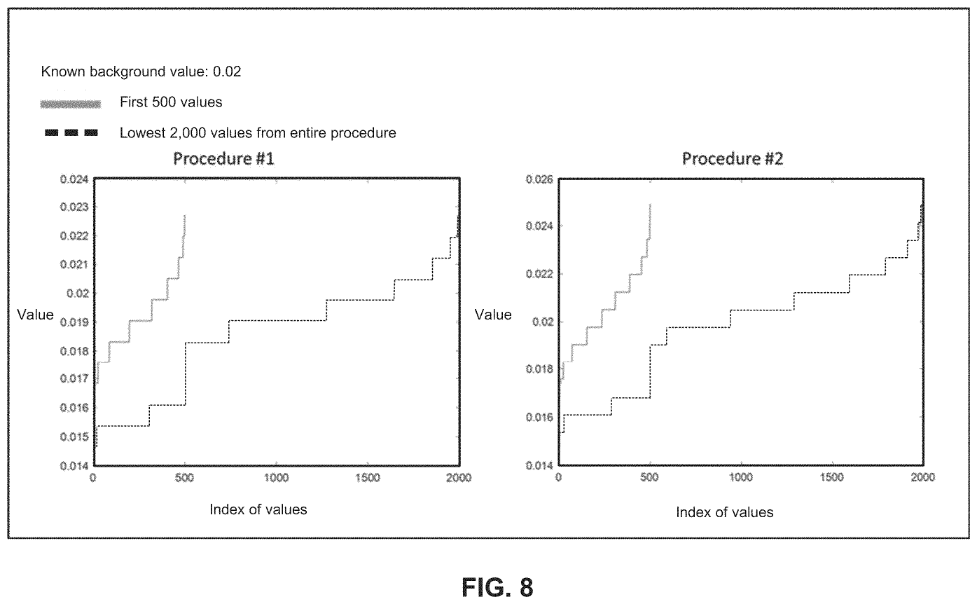

FIG. 8 illustrates an example of detected fluorescence values from two optical-scanning procedures sorted according to the workflow of FIG. 7. In FIG. 8, a solid line shows the first 500 detected fluorescence values from each optical-scanning procedure (sorted in order of smallest to largest), and a dashed line shows the 2,000 lowest detected fluorescence values from each optical-scanning procedure (sorted in order of smallest to largest). The example of FIG. 8 shows that at least one algorithm provides accurate measurements usable with both small and large samples. In procedure #1, the first 500 values are digitized and sorted from a lowest value of about 0.017 to a highest value of about 0.023; similarly, the lowest 2000 values from the entire procedure are digitized and sorted from a lowest value of about 0.0145 to a highest value of about 0.0225. Similar observations are evident in procedure #2 with slightly different values. Since the known (or presumed) background value was 0.02, it is evident that the algorithms disclosed herein can provide a more accurate determination of the background fluorescence value without overestimating or underestimating the background signals. In other words, to obtain a more accurate result in calculating the Background NIRAF value, the use of an average of the lowest 1% of total NIRAF values in the entire operation gets approximately the same results as using the lowest average NIRAF value across all frames of the operation. 2000 NIRAF values is approximately 1% of a typical MMOCT pullback operation, so FIG. 8 shows that the calculation of the Background NIRAF value is applicable to other applications. Advantageously, the measurement of the background fluorescence value can be done within the environment where the instrument is used, thereby minimizing the probability of background miscalculation.

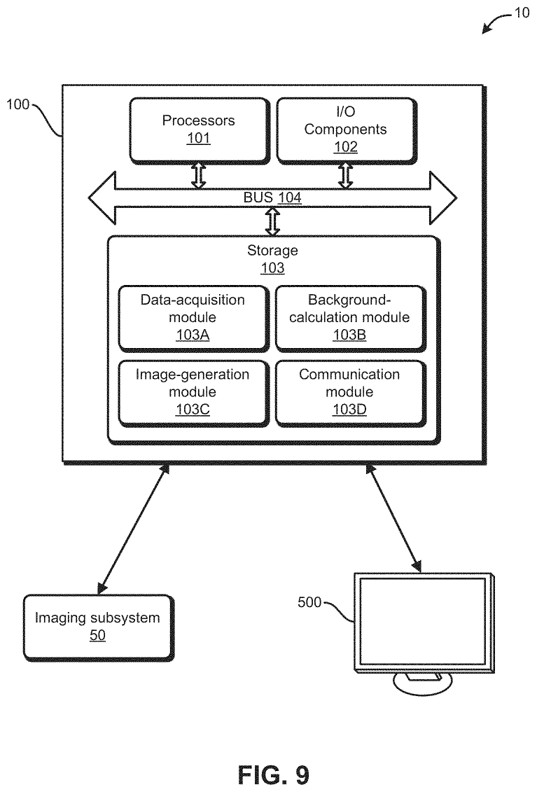

FIG. 9 illustrates an example embodiment of a medical-imaging system. The medical-imaging system 10 includes an image-processing device 100, which is a specially-configured computing device; an imaging subsystem 50; and a display device 500.

The image-processing device 100 includes one or more processors 101, one or more I/O components 102, and storage 103. Also, the hardware components of the image-processing device 100 communicate via one or more buses 104 or other electrical connections. Examples of buses 104 include a universal serial bus (USB), an IEEE 1394 bus, a PCI bus, an Accelerated Graphics Port (AGP) bus, a Serial AT Attachment (SATA) bus, and a Small Computer System Interface (SCSI) bus.

The one or more processors 101 include one or more central processing units (CPUs), such as microprocessors (e.g., a single core microprocessor, a multi-core microprocessor); one or more graphics processing units (GPUs); one or more application-specific integrated circuits (ASICs); one or more field-programmable-gate arrays (FPGAs); one or more digital signal processors (DSPs); or other electronic circuitry (e.g., other integrated circuits). The I/O components 102 include communication components (e.g., a GPU, a network-interface controller) that communicate with the display device 500, the imaging subsystem 50, a network (not shown), and other input or output devices (not illustrated), which may include a keyboard, a mouse, a printing device, a touch screen, a light pen, an optical-storage device, a scanner, a microphone, a drive, a joystick, and a control pad.

The storage 103 includes one or more computer-readable storage media. As used herein, a computer-readable storage medium refers to a computer-readable medium that includes an article of manufacture, for example a magnetic disk (e.g., a floppy disk, a hard disk), an optical disc (e.g., a CD, a DVD, a Blu-ray), a magneto-optical disk, magnetic tape, and semiconductor memory (e.g., a non-volatile memory card, flash memory, a solid-state drive, SRAM, DRAM, EPROM, EEPROM). The storage 103, which may include both ROM and RAM, can store computer-readable data or computer-executable instructions.

The image-processing device 100 additionally includes a data-acquisition module 103A, a background-calculation module 103B, an image-generation module 103C, and a communication module 103D. A module includes logic, computer-readable data, or computer-executable instructions. In the embodiment shown in FIG. 9, the modules are implemented in software (e.g., Assembly, C, C++, C#, Java, BASIC, Perl, Visual Basic, etc.). However, in some embodiments, the modules are implemented in hardware (e.g., customized circuitry) or, alternatively, a combination of software and hardware. When the modules are implemented, at least in part, in software, then the software can be stored in the storage 103. Also, in some embodiments, the image-processing device 100 includes additional or fewer modules, the modules are combined into fewer modules, or the modules are divided into more modules.

The data-acquisition module 103A includes instructions that cause the image-processing device 100 to obtain fluorescence-detection data, which include detected fluorescence values, from the imaging subsystem 50. For example, some embodiments of the data-acquisition module 103A include instructions that cause the image-processing device 100 to perform at least some of the operations that are described in block B210 in FIG. 2, in block B310 in FIG. 3, in block B410 in FIG. 4, in blocks B510 and B540 in FIG. 5, and in block B705 in FIG. 7.

The background-calculation module 103B includes instructions that cause the image-processing device 100 to calculate a background value of detected fluorescence values. For example, some embodiments of the background-calculation module 103B include instructions that cause the image-processing device 100 to perform at least some of the calculations that are described in block B230 in FIG. 2; in blocks B320-B340 in FIG. 3; in blocks B420-B430 in FIG. 4; in blocks B520, B530, and B550-B580 in FIG. 5; and in blocks B710-B740 in FIG. 7.

The image-generation module 103C includes instructions that cause the image-processing device 100 to generate one or more images (e.g., fluorescence images, multimodal images) based on obtained fluorescence-detection data, which include detected fluorescence values; on background fluorescence values; and, in some embodiments, on other image data (e.g., OCT data). For example, some embodiments of the image-generation module 103C include instructions that cause the image-processing device 100 to perform at least some of the adjusting and generating operations that are described in blocks B240-B250 in FIG. 2.

The communication module 103D includes instructions that cause the image-processing device 100 to communicate with other devices, such as the display device 500 and other computing devices. For example, some embodiments of the communication module 103D include instructions that cause the image-processing device 100 to perform at least some of the display operations that are described in block B260 in FIG. 2.

### Use Case Example

A use case scenario for devices, systems, and methods that perform intravascular fluorescence imaging with improved removal of background NIRAF is described next. As it can be recalled from the above description, FIG. 2 illustrates an example embodiment of an operational flow for intravascular fluorescence imaging or intravascular multimodality imaging (e.g. MMOCT imaging) performed by system 10 shown in FIG. 1. The catheter system 10 can provide NIRAF images as a binary color map of detected plaque features of vessels, and such images can be used by a physician in detecting certain vessel abnormalities, such as plaque.

In system 10, the excitation light source 200 outputs excitation light of approximately 635 nm from a laser diode which is coupled into a single mode (SM) fiber to deliver the excitation light to the PIU 330. From the PIU, the excitation light is delivered to vessel tissue via the catheter's imaging core through the core of a double clad fiber (DCF), and the emitted endogenous fluorescent signal (i.e., auto-fluorescence) induced by the excitation light is collected by the same catheter imaging core through the cladding of the DCF. In a MMOCT system, light from the OCT light source 300 is also delivered and collected through the same imaging core of imaging device 318. In this manner, the system 10 can simultaneously collect an OCT signal and a fluorescence signal, which can be spatially co-registered since both signals are obtained from the same region of the bodily lumen.

At the PIU 330, the auto-fluorescence light is separated from the OCT light by a beam combiner (not shown), and the auto-fluorescence is coupled into a multi-mode fiber. The auto-fluorescence light is next delivered to the detector 326, which in this example is a photomultiplier tube (PMT). The PMT is configured to convert analog electrical signals into a digital data stream of fluorescence-detected values which, in one embodiment, correspond to the intensity of the collected auto-fluorescence (NIRAF intensity). Those skilled in the art will appreciate that other parameters such as fluorescence lifetime, fluorescence wavelength, amplitude and the like can be used to convert analog electrical signals into a digital data stream of fluorescence-detected values.

Fluorescence lifetime (FLT) can be measured in either frequency domain or time domain. The time domain method involves the illumination of a sample (a cuvette, cells in a microscope, or tissue) with a short pulse of light, followed by measuring the emission intensity against time. The FLT is determined from the slope of the decay curve. Several fluorescence detection methods are available for lifetime measurements, of which, time-correlated single photon counting (TCSPC) enables simple data collection and enhanced quantitative photon counting. The frequency domain method involves the sinusoidal modulation of the incident light at high frequencies. In this method, the emission occurs at the same frequency as the incident light accompanied with a phase delay and change in the amplitude relative to the excitation light (demodulation).

In the use case experiment performed by the inventors, the NIRAF intensity (e.g., watts/mm²) was converted to nano-molar (nM) units of concentration to correlate the limit of detection (LOD) of the system's detector with a custom NIRAF phantom. NIRAF phantoms were designed to be imaged in order to evaluate, analyze and calibrate the NIRAF performance of the imaging core when imaging specific bodily lumens. In one implementation a NIRAF phantom was designed to mimic auto-fluorescence signals emitted from typical healthy coronary arteries. The NIRAF phantoms are made by mixing Titanium Dioxide (TiO2) and Qdot 705 fluorescent dye. In this case, a NIRAF phantom having 1.0% TiO2 and the Qdot 705 fluorescent dye are used to generate a binary color map image. The binary color map image was processed from the acquired NIRAF data in a MMOCT software application.

NIRAF signal processing was performed by a combination of hardware and software modules. The hardware module includes an excitation laser source (center wavelength 635 nm), a PMT detector, and a low-pass filter. The software module includes one or more of the software algorithms according to FIGS. 3, 4, 5 and 7 described above for NIRAF signal processing.

In the hardware module, the laser power and the PMT gain are adjusted and calibrated for optimal performance. The laser optical power is adjusted to compensate for optical losses of the system to output at the PIU/catheter interface consistently. To that end, the laser power was set approximately 1 mW; this low power setting is designed to ensure patient safety. The PMT gain is calibrated to obtain consistent NIRAF detection sensitivity even at the low power setting of the laser. The analog signal from the PMT goes through the hardware low-pass filter (an anti-aliasing filter) to prevent aliasing and to satisfy the Nyquist-Shannon sampling theorem. After filtering, the analog signal with voltage values in a range of about −0.5 to 2.5 volts is digitized at 100 kHz with 12-bit analog-to-digital (ADC) converter of the first data-acquisition device 324.

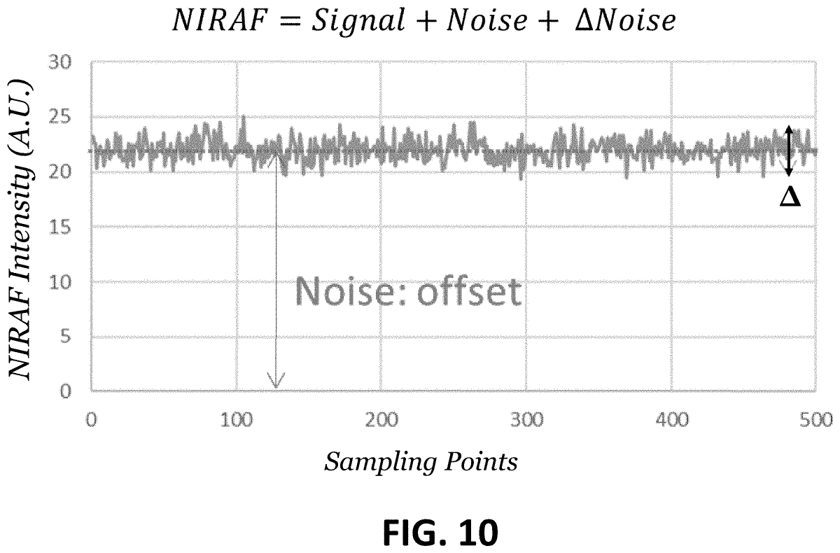

The acquired NIRAF 12-bit data (2¹²=4096 digital values) is reformatted to a signal in a voltage range from 0.0 to 2.5 volts. To that end, the negative values (−0.5 to 0 volts) of the original signal are discarded and replaced with zero values (null values). This reformatted data is saved as raw NIRAF data into a raw-data memory. FIG. 10 illustrates an example of a raw NIRAF data frame (or raw NIRAF data of a single A-line scan) acquired by the imaging core of optical-imaging device 318. The raw NIRAF data is a combination of an amount of random noise (ΔNoise), an amount of background fluorescence (fluorescence noise), and an amount of true fluorescence signal.

From the NIRAF raw data, background noises are removed by subtracting the background offset noise to analyze only the true NIRAF signal elevations (peaks) coming from the sample. The origin of the background noise in the instrument is mostly due to fluorescence from double clad fibers from a beam combiner in the PIU, and from the catheter imaging core (e.g., double clad fiber and distal optics). However, even after factoring the instrument background noise, the raw NIRAF data still contains noise coming from the sample itself. The origin of sample noise is mostly due to fluorescence from residual blood or imaging media (e.g., flushing media or tagging agents (fluorophores) not binded to the intended molecules of the sample). Since the imaging core continuously scans the sample (e.g., the vessel wall) with a rotating beam, the background fluorescence is seen as random noise at the bottom of the true NIRAF signal elevations coming from the sample. Therefore, to remove the background fluorescence due to sample noise, the inventors herein propose a novel solution of using a single background fluorescence value (a background fluorescence threshold) to remove the background fluorescence from the entire pullback signal.

Advantageously, according to at least one embodiment (see FIGS. 3-5 and 7), the single background NIRAF value is calculated from N detected fluorescence values. The N detected fluorescence values can include a portion a full NIRAF frame (e.g., a portion of first frame), or can include all values from one single NIRAF frame, or can include low fluorescence values from multiple NIRAF frames, or can include all fluorescence values from some NIRAF frames (e.g., the first frame and last frame) of a complete pullback procedure. Regardless of the manner in which the single background NIRAF value is calculated, a single detected background fluorescence value is used as a threshold for removing the background noise from the whole pullback. In one example, each background NIRAF frame contains 500 data samples (N fluorescence data values). The background NIRAF value is calculated by finding a central tendency (e.g., an average) of the 500 background NIRAF samples to minimize random noises.

The background fluorescence value is chosen from the lowest averaged NIRAF frame of the pullback data. If the whole pullback region of the lumen (50 mm to 80 mm) has high NIRAF signals, the background data could be overestimated. Therefore, to mitigate the potential overestimation of the background NIRAF value, several algorithms are described to choose the lowest background NIRAF value coming from the sample signal.

It is known that the sample NIRAF signals of a complete pullback procedure will contain noise that can be random across the different frames, and therefore the background NIRAF value could be potentially overestimated or underestimated. To avoid this issue, in at least one embodiment, a threshold window is set above the noise level to ensure the detected signals are coming from the sample (tissues or phantom). If the signals are below the threshold window, the NIRAF values are considered as no signals (zero or null values); if the signals are above the threshold window, the NIRAF value is considered as a true fluorescence signal coming from a sample. But to avoid overestimating or underestimating the background NIRAF value, the threshold window can be dynamically adjusted for every pullback.

In an experiment for the use case scenario a NIRAF background frame is chosen from the smaller averaged NIRAF value of the following two cases. Case 1: a NIRAF background frame is acquired when/right after catheter engagement. In this case, the catheter is outside of a bodily lumen, and if strong ambient light illuminate to the catheter imaging window, the NIRAF background value could be overestimated. Case 2: a NIRAF background frame is chosen from the lowest averaged NIRAF frame of the pullback data of a complete pullback procedure. If the whole pullback region (50 mm or 80 mm) has high NIRAF signals, the background data could be overestimated. Since the true NIRAF signal would ideally need to be zero when acquiring the NIRAF background noise (as defined by the equation in FIG. 10), the process to select the smaller value from Case 1 and Case 2 helps mitigate the potential overestimation of NIRAF background value for each case.

In one embodiment, to evaluate whether detected NIRAF signals are effective to compare with background noise level, a threshold window as shown in Expression (1) was established.

$$Threshold window = \{ \begin{matrix}5 * Std & when 5 * std \leq 50 * \frac{4096}{3000} \\ 50 * \frac{4096}{3000} & when 5 * std > 50 * \frac{4096}{3000}\end{matrix} \qquad Expression ( 1 )$$

In Expression (1), the threshold value is calculated using 90% of the lowest data values (the lowest 450 points) from a 500 A-line background NIRAF frame. In addition, the top 10% (highest 50 points) of the background NIRAF frame was used to derive the standard deviation (std) in order to eliminate potential overestimated signals. The potential overestimations could be ambient light, catheter dust or other objects caused by having the catheter too close to the sample or touching the sample when acquiring the NIRAF data.

In addition to eliminating 10% of highest data (50 points) above, the threshold value stays under 50 mV*4096/3000 as maximum threshold value. The NIRAF signal elevations from 0 nM to 5 nM at distance of 0 mm (touching the sample) can estimate 262.5 (=52.5*5) mV. Considering a distance correction factor of about 0.375 at a distance of 4 mm, the raw NRIAF value can be 98.4 (262.5*0.375) mV as a worst case scenario. Raw NIRAF data of 50 mV is below of that expected minimum 5 nM value (98.4 mV). 4096/3000 is the conversion factor from mV to digital unit, and nM conversion factor of 52.5 mV/nM.

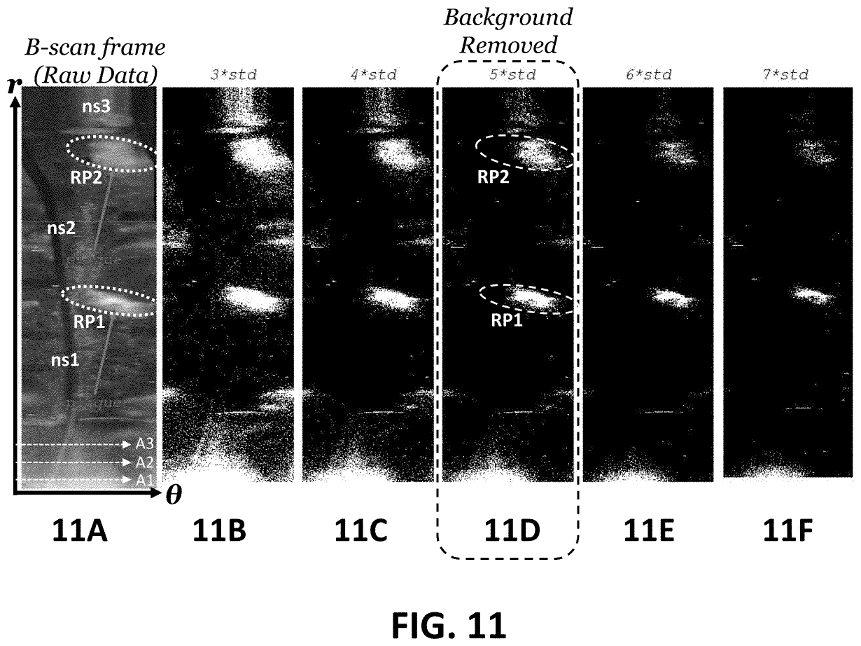

Based on the foregoing considerations, a factor of 5*std was determined to eliminate most of Background NIRAF data without removing true NIRAF data. If this factor is too high, the system can reliably eliminate the Background NIRAF data as noise. However, the LOD (limit of detection) of the system will decrease. The factor of 5*std was optimized with NIRAF ex-vivo data, as shown in FIG. 11. FIG. 11 illustrates a series of images 11A, 11B, 11C, 11D, 11E, and 11F generated from an ex-vivo data set of NIRAF signals, where a multiple threshold factor was used to remove background fluorescence. In FIG. 11, image 11A is a B-scan frame including raw data collected by the imaging system 10. A B-scan frame is formed by grouping a plurality of A-lines (A0, A1, A2, A3 . . . An) next to each other to form a rectangular image where the horizontal axis (x-axis) corresponds to the angle theta (θ) and the vertical axis (y-axis) corresponds to the radius (r) in polar coordinates of an intravascular image acquired by an imaging device 306 of FIG. 1.

In the B-scan of image 11A, a bright fluorescence region can be observed close to the catheter axis (at the bottom rows of the image). This bright fluorescence region near the catheter axis corresponds to instrument noise or background fluorescence coming from the imaging device (optical fiber of imaging core, distal optics, catheter sheath, etc.). As the radius (r) of the image expands away from the catheter axis (in the imaging depth direction), a first region of fluorescence RP1 (e.g., originated from plaque) and a second region of fluorescence RP2 (e.g., originated from plaque or other fluorophore) can be observed at differing levels of brightness. In addition, background fluorescence artifacts or noise (ns1, ns2, ns3) can be observed throughout the image in the direction of depth of imaging. When background fluorescence removal is applied to the B-scan image, the result is shown in background-removed images 11B through 11F.

Images 11B, 11C, 11D, 11E, and 11F show the same data of image 11A after background fluorescence removal has been applied according to one or more of the algorithms disclosed herein. In all of the background-removed images, the “white” regions of the image represent tissue fluorescence above the threshold background fluorescence value, and the “black” regions of the image represent removed fluorescence which was below the threshold background fluorescence value. By analyzing all of the background-removed images, the inventors herein determined that image 11D, where a threshold window with a 5*std (5-sigma) was applied, satisfies the requirement that background fluorescence removal neither overestimates nor underestimates the Background NIRAF value. Moreover, the inventors confirmed that the threshold window can be dynamically adjusted to calculate a threshold value appropriate for every pullback procedure during the course of normal data collection and within the actual environment where the measurements are occurring.

The present disclosure, as used for a multimodality OCT-NIRAF (MMOCT) imaging system, allows the background fluorescence to be calculated without the use of another piece of equipment. The system can be configured to make calculations of background fluorescence during the normal course of optical-scanning operations instead of in a separate operation. This can ensure that the correct background fluorescence value is calculated, and background removal is applied to the results being displayed by the application. Since the background calculation and removal takes place during the normal course of operations, the system can remove the potential for an incorrect background calculation due to environment. The algorithms disclosed herein work well on small samples (e.g., on one frame of 500 A-lines of an MMOCT image) or on large samples (e.g., the entire pullback) reduced to a percentage of the lowest values or an average thereof to more expeditiously calculate a threshold background fluorescence value. However, as mentioned above, the algorithms would work as implemented even if there is only 1 A-line per frame. As long as we had at least 2 frames with the lower value frame serving as the background fluorescence value used as a threshold. This is true when the algorithm uses an average as the central tendency.

The scope of the claims is not limited to the above-described embodiments or to the specific use case scenario, as various modifications, combinations, and equivalent arrangements of the embodiments are applicable. The scope of the following claims is to be accorded the broadest interpretation so as to encompass all such modifications and equivalent structures and functions.

Any patent, pre-grant patent publication, or other disclosure, in whole or in part, that is said to be incorporated by reference herein is incorporated only to the extent that the incorporated materials do not conflict with standard definitions or terms, or with statements and descriptions set forth in the present disclosure. As such, and to the extent necessary, the disclosure as explicitly set forth herein supersedes any conflicting material incorporated by reference.

What is claimed is:

1. A method for accurately quantifying fluorescence emitted from within a bodily lumen, the method comprising:
scanning a bodily lumen with an imaging catheter that transmits a light of wavelength capable of stimulating emission of fluorescence from within different regions of the bodily lumen;
collecting a plurality of fluorescence data values corresponding to the fluorescence emitted from the different regions within the bodily lumen;
grouping the plurality of fluorescence data values into one or more frames,
calculating a central tendency of fluorescence data values in each of the one or more frames;
assigning the central tendency of one of the one or more frames as a background fluorescence threshold;
adjusting the fluorescence data values of the one or more frames based on the background fluorescence threshold, thereby generating adjusted fluorescence data values; and
generating an image based on the adjusted fluorescence data values.

2. The method according to claim 1,
wherein generating the image includes generating an image of the bodily lumen based on fluorescence data values that are larger than a value of the background fluorescence threshold.

3. The method according to claim 1, further comprising:
assigning the central tendency of one of the one or more frames as a background fluorescence threshold includes assigning the lowest central tendency among the central tendencies of all frames as the background fluorescence threshold.

4. The method according to claim 1,
wherein the one or more frames includes one B-scan frame, and the plurality of fluorescence data values respectively corresponds to a plurality of A-lines of the one B-scan frame collected by the imaging catheter in one full revolution within the bodily lumen,
wherein collecting the plurality of fluorescence data values includes collecting one fluorescence data value for each of the A-lines, and
wherein calculating the central tendency includes calculating a mean of the fluorescence data values corresponding to at least a portion of the A-lines in the one B-scan frame, or
wherein calculating the central tendency includes calculating a mean of the fluorescence data values corresponding to all of the A-lines in the one B-scan frame.

5. The method according to claim 1,
wherein the one or more frames includes a plurality of B-scan frames collected by the imaging catheter during an entire pullback within the bodily lumen.

6. The method according to claim 5,
wherein collecting the plurality of fluorescence data values includes collecting one fluorescence data value for each of the A-lines in 1% of the plurality of B-scan frames of the entire pullback, and
wherein calculating the central tendency includes calculating a mean of the fluorescence data values in the 1% of the plurality of B-scan frames.

7. The method according to claim 1,
wherein the one or more frames includes one or more B-scan frames acquired by the imaging catheter in a region of low or no fluorescence within the bodily lumen.

8. The method according to claim 7,
wherein collecting the plurality of fluorescence data values includes collecting one fluorescence data value for each of the A-lines of the one or more B-scan frames, and
wherein calculating the central tendency includes calculating an average of the fluorescence data values for each of the one or more B-scan frames, and selecting the lowest average among the averages for the one or more B-scan frames.

9. The method according to claim 1,
wherein the one or more frames includes a first B-scan frame acquired by the imaging catheter from a first region within the bodily lumen,
wherein collecting the plurality of fluorescence data values includes collecting one fluorescence data value for each of the A-lines of the first B-scan frame acquired from the first region, and
wherein calculating the central tendency includes calculating an average of the fluorescence data values for the first B-scan frame.

10. The method according to claim 9,
wherein the one or more frames further includes a second B-scan frame acquired by the imaging catheter from a second region different from the first region within the bodily lumen,
wherein collecting the plurality of fluorescence data values further includes collecting one fluorescence data value for each of the A-lines of the second B-scan frame acquired from the second region,
wherein calculating the central tendency further includes calculating an average of the fluorescence data values for the second B-scan frames, and
wherein assigning the central tendency of one of the one or more frames as a background fluorescence threshold includes assigning the lowest central tendency among the central tendencies of the first B-scan frame and the second B-scan frame as the background fluorescence threshold.

11. A catheter-based imaging system for quantifying fluorescence emission from a bodily lumen, the system comprising:
an imaging catheter that scans a bodily lumen with light of wavelength capable of stimulating emission of fluorescence from within the bodily lumen; and
a processor configured to:
collect a plurality of fluorescence data values corresponding to the fluorescence emitted from different regions within the bodily lumen;
group the plurality of collected fluorescence data values into one or more frames;
calculate a central tendency of fluorescence data values in each of the one or more frames;
assign the central tendency of one of the one or more frames as a background fluorescence threshold;
adjust the fluorescence data values of the one or more frames based on the background fluorescence threshold, thereby generating adjusted fluorescence data values; and
generate an image based on the adjusted fluorescence data values.

12. The system according to claim 11,
wherein the fluorescence data values correspond to one or more of an intensity, an amplitude, and a lifetime of the detected fluorescence light.

13. The system according to claim 11,
wherein the fluorescence data values include N detected fluorescence values, where N is a positive integer greater than 2, and
wherein the processor calculates one or more of an average, a mean, or a median as the central tendency of the at least part of the fluorescence data values.

14. The system according to claim 11,
wherein the processor is further configured to:
discard fluorescence data values that are equal to or lower than the background fluorescence threshold; and
generate the image based on fluorescence data values that are larger than a value of the background fluorescence threshold.

15. The system according to claim 11,
wherein the processor assigns the central tendency of the at least part of the fluorescence data values as the background fluorescence threshold of all of the fluorescence data values.

16. The system according to claim 11,
wherein the processor (i) receives the first N detected fluorescence values, (ii) calculates a mean of the first N detected fluorescence values.

17. The system according to claim 16,
wherein the processor sets the mean of the first N detected fluorescence values as the background fluorescence threshold for a first frame of a pullback procedure.

18. The system according to claim 16,
wherein the processor sets the mean of the first N detected fluorescence values as the background fluorescence threshold for all frames of an entire pullback procedure.

19. The system according to claim 11,
wherein the processor acquires detected fluorescence values of a plurality of A-scan lines that were generated based on the fluorescence light collected by the imaging catheter while scanning the bodily lumen;

20. The system according to claim 11,
wherein the processor sorts the central tendencies of all frames from lowest to highest, and assigns the lowest central tendency among the central tendency of the one or more frames as the background fluorescence threshold for all frames.
